(19) United States
(12) Patent Application Publication
(10) Pub. No.: US 2022/0252624 A1
(43) Pub. Date: Aug. 11, 2022

(54) METHOD FOR DETERMINING THE RISK OF A THROMBOEMBOLIC EVENT

(71) Applicant: ACADEMISCH MEDISCH CENTRUM, Amsterdam (NL)

(72) Inventors: Cornelis VAN 'T VEER, Utrecht (NL); Anja MAAG, Amsterdam (NL)

(21) Appl. No.: 17/618,796

(22) PCT Filed: Jun. 15, 2020
(86) PCT No.: PCT/EP2020/066526

§ 371 Date: Dec. 13, 2021

(30) Foreign Application Priority Data
Jun. 15, 2019 (EP) 19180416.0

(51) Int. Cl. G01N 33/86

(52) U.S. Cl. CPC G01N 33/86; G01N 2333/745; G01N 2333/96444; G01N 2800/226

(57) ABSTRACT

The invention relates to a method of determining the bleeding risk of a subject comprising:
a. contacting a first sample from the subject with an activation mixture comprising (I) TIX-5, (II) factor Xa or an activation agent for activating directly or indirectly the conversion of factor X to factor Xa, and (III) a phospholipid mixture,
b. determining the value of a coagulation function parameter of said sample,
c. comparing the coagulation function parameter with a control value,
d. determining the bleeding risk based on a comparison between the value of said coagulation function parameter of said first sample and said control value.

### FIELD OF THE INVENTION

The present invention relates to the field of thromboembolic diseases or disorders. More specifically, it relates to methods for determining whether a subject, particularly a human subject, is at risk of developing a thromboembolic event or a major bleeding.

### BACKGROUND

Thromboembolic diseases or disorders with a risk of thrombotic complications such as atrial fibrillation (AF) are treated with anticoagulant therapy. Yet the anticoagulant therapy comes with an associated risk of bleeding that can be fatal. Several bleeding prediction scores have been described: HAS-BLED, ATRIA, HEMORR2HAGES and ORBIT. Of these, only HAS-BLED considers quality of anticoagulation control amongst vitamin K antagonist (VKA) users. The HAS-BLED score is however far from perfect (predictive value c statistics for bleeding only 0.50-0.68) and is in high need for improvement of a method for predicting the risk of bleeding or the occurrence of a thromboembolic event.

WO 02/34109 discloses a method whether a patient is hypercoagulable, hypocoagulable or normal in a single test on a sample from the patient. This method uses thrombomodulin as anticoagulant. A disadvantage of using thrombomodulin is that this only improves the detection of hypercoagulation and potential trombotic risks related to deficiencies in the thrombomodulin dependent anticoagulant protein C (PC) pathway. The authors of WO 02/34109 A2 state on page 19, line 18-20 that thombomodulin is not essential to obtain discrimination between a hypocoagulable plasma and a normal pool plasma. There remains a need for tests which are more sensitive to detect whether a patient is hypocoagulable.

### SUMMARY OF THE INVENTION

The present invention is based on the surprising finding that by addition of TIX-5 to plasma samples of VKA patients, a strong association was observed with bleeding in a Calibrated-Automated-Thrombogram (CAT) assay. This is surprising, as in Bloemen et al no differences were found in plasma CAT parameters or INR values in patients taking vitamin K antagonists (Bloemen S, Zwaveling S, ten Cate H, ten Cate-Hoek A, de Laat B (2017) PLoS ONE 12(5): e0176967: see line 10 of the abstract, and data Table 3 (PPP 5 pM TF and PPP 1 pM TF)). Using a CAT assay wherein plasma was activated by 20 pM Tissue Factor in the presence of TIX-5, the inventors observed remarkably long lagtimes in the plasma of patients taking vitamin K antagonists (VKA), which displayed major bleeding during follow up compared to patients on VKA that did not bleed (FIG. 9). This increase in lagtime was associated with a 2.05-fold increase in the risk of bleeding for patients with a longer lagtime (Table 1a).

Furthermore, a high ratio of the lagtime measured in the presence of TIX-5 and the lagtime without TIX-5 was associated with a 1.59-fold increase of risk of bleeding (Table 1a). Moreover, a low total amount of thrombin generated (area under the curve, ETP) in the presence of TIX-5 was associated with a 1.6-fold increase in the risk of bleeding.

Addition of TIX-5 to a Calibrated-Automated-Thrombogram (CAT) assay thus changes this plasma CAT assay from a non-predictive assay to a clinically relevant predictive assay.

Therefore, the invention provides a method of determining the bleeding risk of a subject comprising:
a. contacting a first sample from the subject with an activation mixture comprising (I) TIX-5, (II) factor Xa or an activation agent for activating directly or indirectly the conversion of factor X to factor Xa, and (III) a phospholipid mixture,
b. determining the value of a coagulation function parameter of said first sample,
c. comparing the value of said coagulation function parameter with a control value,
d. determining the bleeding risk based on a comparison between the value of said coagulation function parameter of said first sample and said control value.

In a preferred embodiment, said coagulation function parameter is selected from: coagulation time, the amount thrombin, the lagtime, the time to peak of thrombin, the maximal velocity of thrombin generation and the thrombin peak height.

In a preferred embodiment, said coagulation function parameter is the coagulation time (Ct), wherein an increase of Ct compared to a control value indicates a higher risk of a bleeding.

It is further preferred that said coagulation time is determined by measuring the time between the contacting of the activation mixture with said first sample and the onset of coagulation, thereby calculating the coagulation time (Ct). In a further preferred embodiment, the method of the invention further comprises:

a. contacting a second sample from the subject with an activation mixture comprising (I) factor Xa or an activation agent for activating directly or indirectly the conversion of factor X to factor Xa, and (II) a phospholipid mixture and (III) without TIX-5,

b. measuring the time between the contacting of the activation mixture with said second sample and the onset of coagulation, thereby calculating the coagulation time of said second sample (Ct2), wherein an increase in Ct compared to Ct2 indicates a higher risk of a bleeding.

Preferably, said comparison comprises determining the ratio between Ct and Ct2, wherein an increase in the ratio between Ct and Ct2 indicates a higher risk of bleeding.

In another preferred embodiment, said coagulation function parameter is the amount of thrombin, wherein a risk of bleeding is indicated when the amount of thrombin in said first sample is lower than a thrombin control value.

In a preferred embodiment, TIX-5 is used in a concentration of more than 1, 2, 3 μM, preferably about 4 μM.

In a preferred embodiment of the method according to the invention said an activation agent is Tissue Factor (TF). Preferably, said activation agent has a concentration of more than 5, 10 or 20 pM. Preferably, the concentration of TF should not be higher than 100 pM. Without wishing to be bound by theory, the inventors believe that such concentration would result in a very short lagtime and overpower the balancing pro- and anti-coagulant processes that rule factor V activation in the initiation phase of thrombin generation (van't Veer et al J Biol Chem 1997; 272:4367-4377, and Schuijt et al Circulation 2013; 128:254-266) which would hamper the sensitivity of the coagulation test with TIX-5.

It is preferred that said subject is in need of an anti-coagulant therapy. In a preferred embodiment, said subject is a patient treated using an anticoagulant therapy, preferably a vitamin K antagonist anticoagulant therapy (VKA). Preferably, said subject is a human.

In preferred embodiments, said sample comprises plasma or whole blood. In other preferred embodiments, said sample is an anticoagulant treated plasma sample.

The invention further provides a kit which is suitable for carrying out the method of the invention, comprising:

a. TIX-5,

b. TF in a concentration of 10 pM or higher, and

c. a phospholipid mixture.

The invention further provides the use of TIX-5 in a coagulation assay on an anticoagulant treated plasma sample. Said assay is preferably a CAT assay.

### BRIEF DESCRIPTION OF THE FIGURES

FIG. 1 shows the effect of TIX-5 on thrombin generation in a CAT assay on normal pool citrated plasma (pooled plasma of 60 healthy volunteers). In brief, thrombin generation was measured according to a standard protocol using a Fluoroskan Ascent (Thermolabsystems, Helsinki, Finnland) 96-wells fluorometer. Eighty (80) μL plasma, 20 μL relipidated tissue factor (final concentration 5 pM) in 4 μM (final concentration) phospholipid vesicles consisting of 20% phosphatidylserine/20% phosphatidylethanolamine/60% phosphatidylcholine, with either 10 μL vehicle control (PBS, closed symbols) or 10 μL TIX-5 diluted in PBS (final concentration 4 μM, open symbols) were added into a 96 well round bottom plate and mixed. The 96 well plate with the mixtures was heated for 10 minutes at 37° C. in the fluorometer and thrombin generation was started by addition of 20 μL FluCa fluorogenic substrate/calcium solution (Trombinoscope by, STAGO) to each well (t=0) which starts the thrombin generation reaction by recalcification of the citrated plasma. Thrombin activity was determined by cleavage of the fluorogenic substrate detected by the fluorometer every 20 seconds for 60 minutes. The fluorescence was analysed and converted to thrombin concentration in nM using the Thrombinoscope (STAGO) software that generates the thrombin generation curves based on a calibrated thrombin standard ran in parallel. Thrombin is appointed as FIIa in nM on the y-axes. The software also calculates the lagtime, the maximal velocity of thrombin generation in nM/min, peak height in nM and time to peak (ttPeak) in minutes, and the total amount of thrombin generated defined as endogenous thrombin potential (ETP) in nM*minute of thrombin generation in the plasma sample with vehicle and with TIX-5.

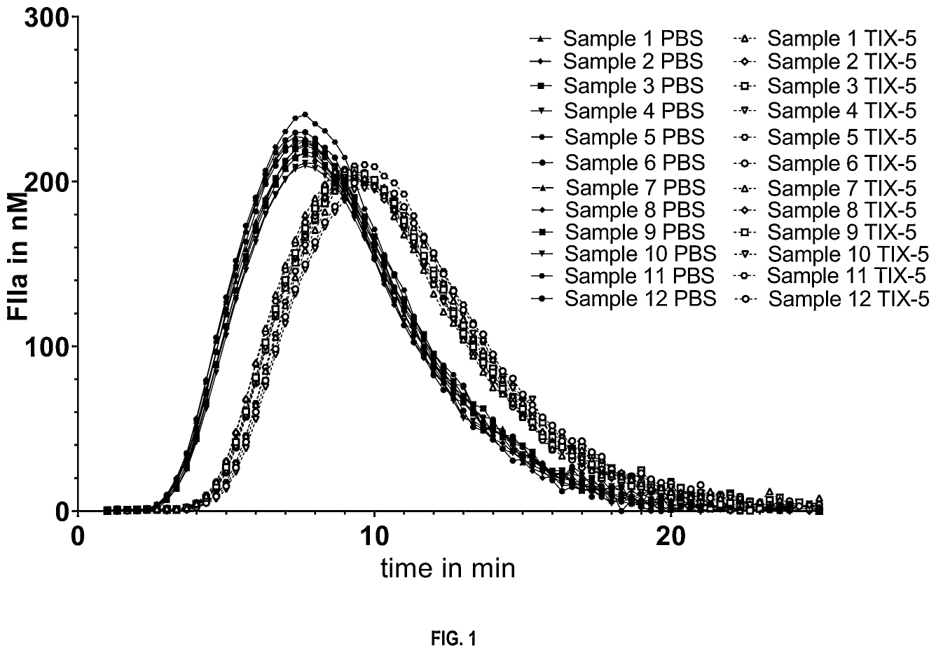

FIG. 2.a shows that the lagtime of thrombin generation is increased in the presence of TIX-5 by 40% in normal pool plasma. When the ratio is calculated for the lagtime+TIX-5/lagtime+vehicle this lagtime ratio is increased to 140% as shown in FIG. 2b. The only thrombin generation parameter that really changes in the presence of TIX-5 in normal pool plasma is the lagtime. FIG. 2b shows that the TIX-5/vehicle ratio for other parameters such as ETP, peak and velocity does not change, i.e, are around 100%.

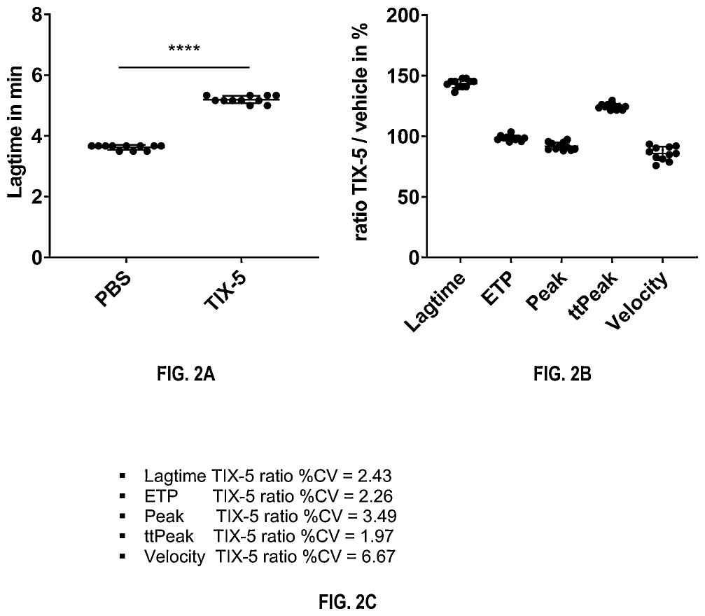

FIG. 2c shows the intra assay variability of the TIX-5 effect on the different parameters of thrombin generation using normal pool plasma.

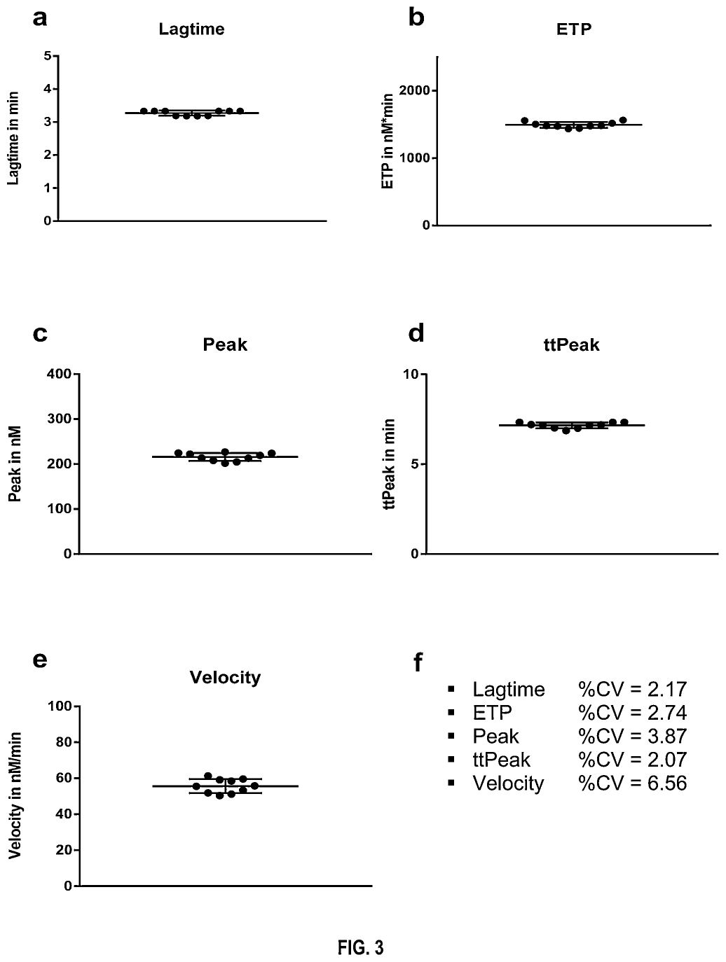

FIG. 3 shows the intra assay variability of the CAT assay started with 5 pM TF, two normal pool plasma samples were tested on different days and plates in subsequent assays.

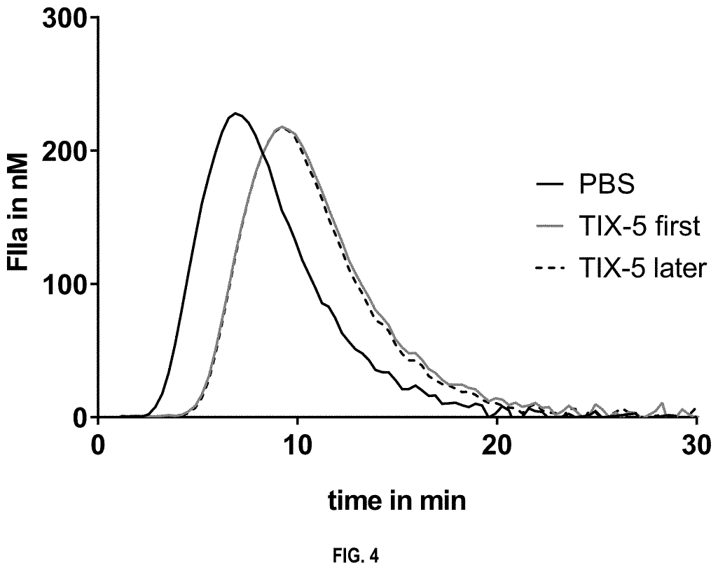

FIG. 4 shows a CAT assay in which it was determined whether the order of pipetting the reagents influences the TIX-5 effect on normal pool plasma. For this the samples were in a 96 well plate according to the general protocol and as described above. Here we either, pipetted TIX-5 (4 μM) and the 5 pM relipidated tissue factor reagent into the plate before adding the plasma (grey solid line), or we added TIX-5 as last reagent after the addition of plasma to the 5 pM relipidated tissue factor (dashed black line). After mixing the plate properly the assay was continued as described before. TIX-5 leads to exactly the same prolongation of the lagtime compared to the PBS vehicle control (solid black line) independent of the order of pipetting.

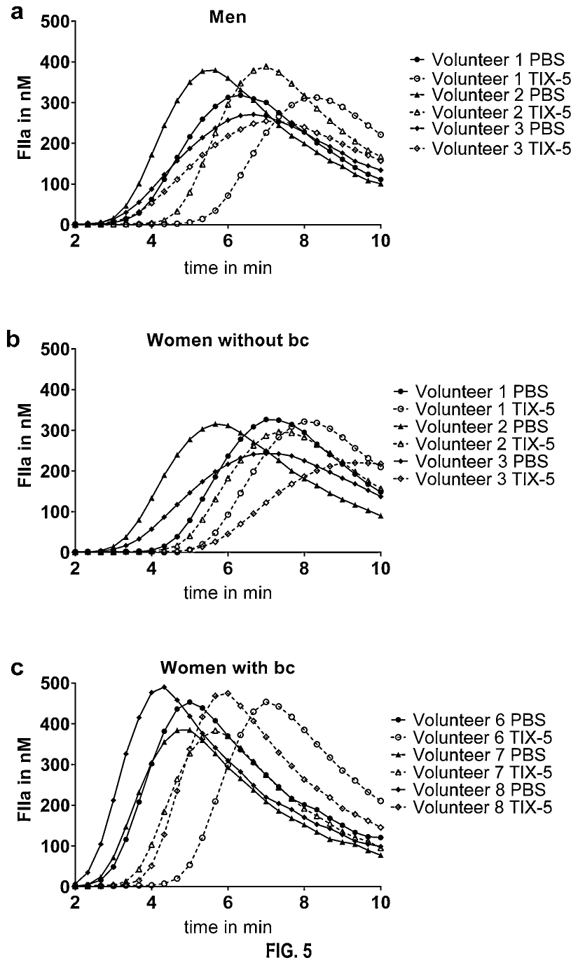

FIG. 5 shows some selected thrombin generation curves activated with 5 pM TF in plasma of individual subjects and the effect of TIX-5. Per individual a different symbol is used, the PBS vehicle sample is depicted by the closed symbol, the corresponding sample with TIX-5 is depicted by the open symbol. Some selected curves are shown for either men (FIG. 5a), women without birth control (FIG. 5b) or women with oral birth control (FIG. 5c) because it is known that oral contraception affects coagulation. The thrombin generation curves in FIG. 5, show that TIX-5 only prolongs the lagtime and time to peak in individual plasmas of healthy volunteers, although the extent of the prolongation differs per individual. Importantly, the difference in the effect of TIX-5 on the lagtime in the individual plasma's is not dependent on the lagtime without TIX-5 or the maximal velocity of thrombin generation. Compare for instance in FIG. 5a graph “Men” and FIG. 5b “Women without bc” the curves with closed and open triangles, diamonds and circles. This suggests that TIX-5 inhibits a unique process in thrombin generation, of which we showed before to be the factor Xa activation of factor V. Thus with this TIX-5 test we can reliably determine the contribution of factor Xa activation of factor V to thrombin generation in an individual plasma.

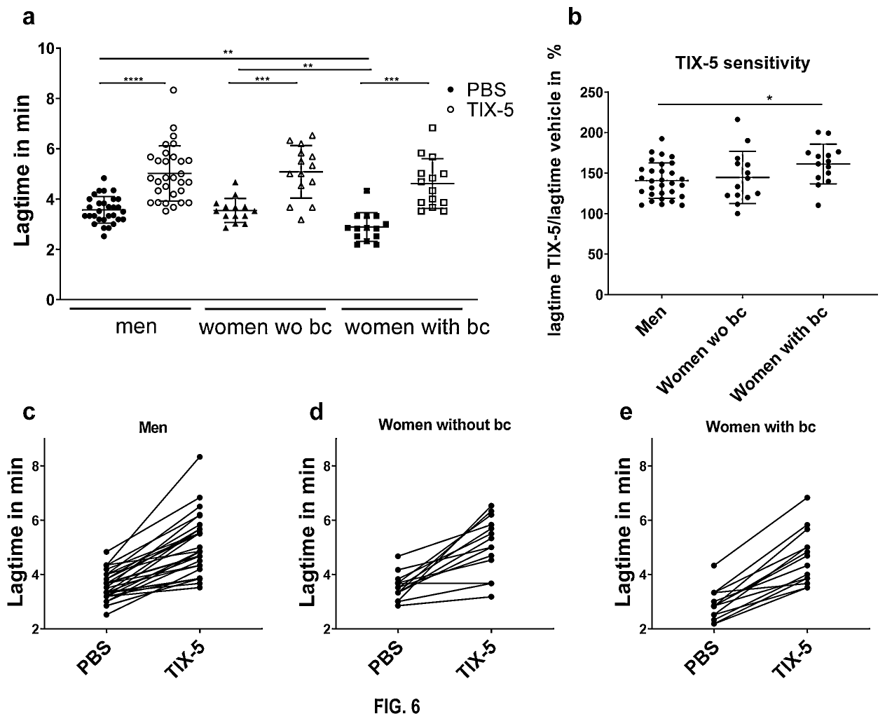

FIG. 6 shows the effect of TIX-5 on the lagtime of thrombin generation determined by CAT initiated by 5 pM TF in plasma of 60 healthy individual subjects among which 30 men, 15 women without birth control (wo bc) and 15 women with oral birth control (with bc). TIX-5 does prolong the lagtime of thrombin generation in each group of the healthy volunteers significantly (FIG. 6a). The extend of the prolongation of the lagtime by TIX-5, or socalled TIX-5 sensitivity differs considerably per individual. This can be concluded from FIG. 6b where the lagtime with TIX-5 is depicted as percentage of the lagtime without TIX-5. As can be seen in some individuals, TIX-5 only increases the lagtime by 10-20% (so 110-120% on the y-axes of FIG. 6b) while in other subjects TIX-5 increases the lagtime by 100% (200% on the y-axes of FIG. 6b). On average the lagtime is prolonged by 40% by TIX-5, which is consistent with the fact that TIX-5 prolonged the lagtime of pooled plasma of 60 healthy volunteers also by 40% as depicted in FIG. 2b.

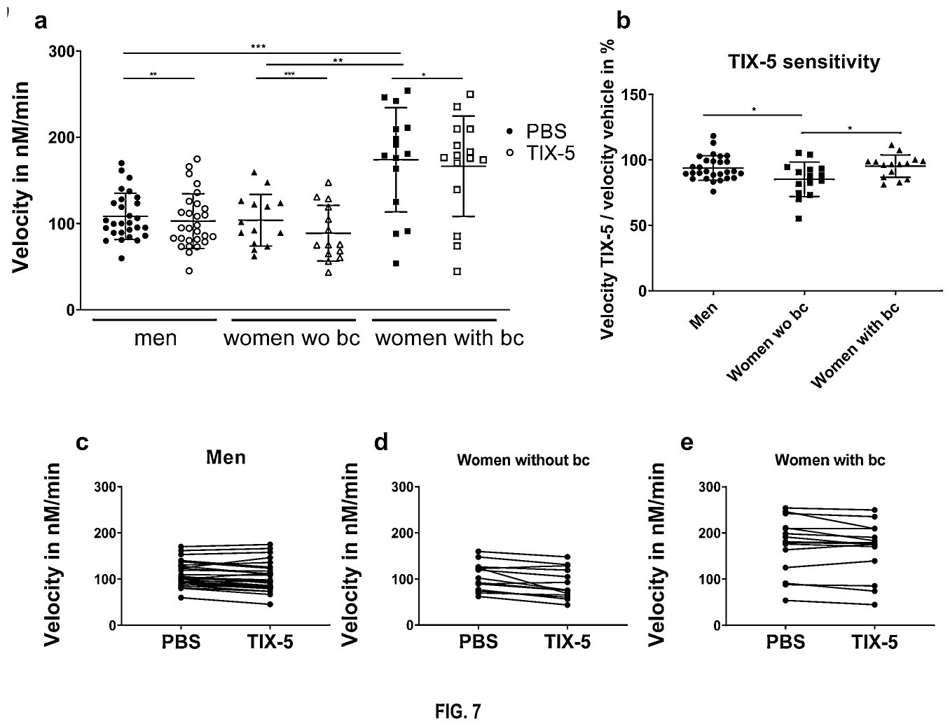

We also find that the lagtime in PBS vehicle samples of Women with oral bc is shorter compared to the lagtime of both Men as well as Women without bc (FIG. 6a), and importantly, that the plasma of Women with oral bc is more sensitive to prolongation of the lagtime by TIX-5 (FIG. 6b).

FIG. 7 shows the effect of TIX-5 on the maximal velocity of thrombin generation. TIX-5 does not affect the maximal velocity of thrombin generation in healthy volunteers. Women using birth control display an increased maximal velocity of thrombin generation; this higher velocity was not affected by TIX-5 (FIG. 7a).

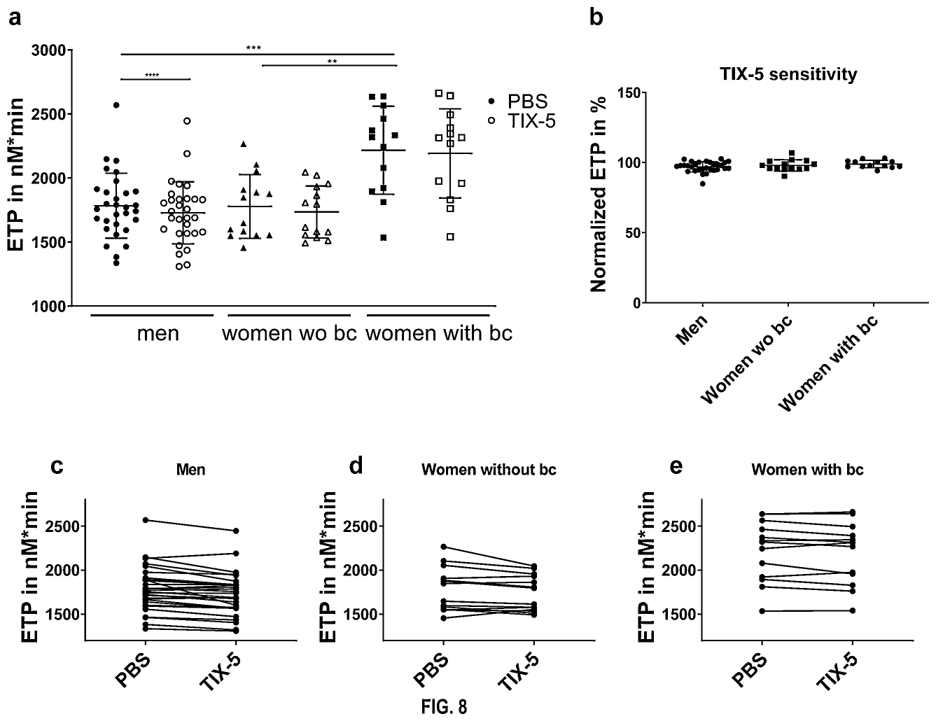

FIG. 8 shows the effect of TIX-5 on the total amount of thrombin generated or socalled endogenous thrombin potential (ETP). TIX-5 has no effect on the ETP in plasma of healthy volunteers (FIG. 8a). The ETP is higher for Women with bc, but this higher ETP is not affected by TIX-5 (FIG. 8a).

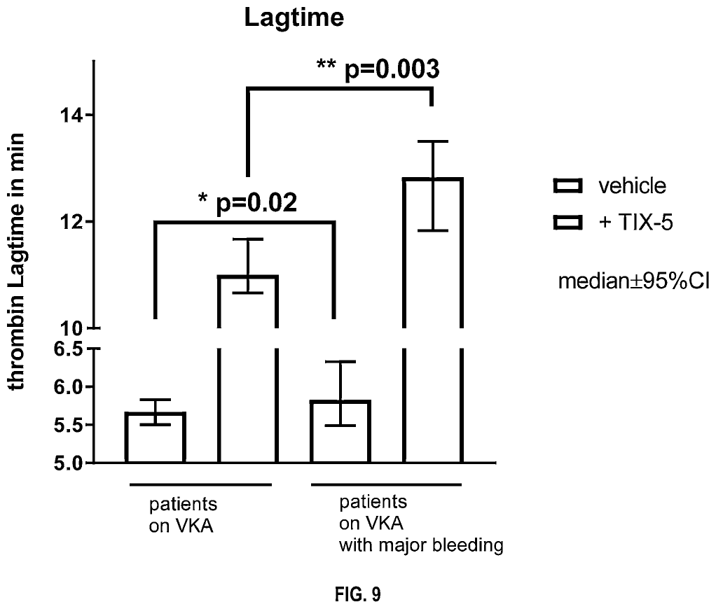

This shows that the sensitivity of tissue factor initiated thrombin generation of healthy individuals to the inhibition of TIX-5, the inhibitor of factor Xa activation of factor V, differs profoundly. This suggest that the contribution of factor Xa activation of factor V to thrombin generation varies importantly between individuals. To investigate whether differences in the contribution of factor Xa activation of factor V is associated with bleeding during anticoagulant therapy we tested the TIX-5 sensitivity of patients with major bleeding of the BLEEDS study (van Rein et al, PLoS ONE 2016; 11:e0164485). In the BLEEDS study plasma was collected of patients after 3 weeks of start with VKA anticoagulant therapy and bleeding while under VKA therapy was monitored. Incidence rate of bleeding was 1.85/100 person years.

FIG. 9 shows the result of the TIX-5 CAT assay initiated with 20 pM TF on stored plasma's of a randomly chosen subcohort of the BLEEDS study (538 plasma's) and compared those to plasmas of the cases with major bleeding (244 patients) while under VKA anticoagulant therapy. Lagtimes of thrombin generation in the presence of TIX-5 (white bars) are significantly longer in patients that displayed major bleeding (FIG. 9) which translates in a significant 2.05-fold increased risk for bleeding (Table 1a) in the quartile with the highest lagtime+TIX-5. Furthermore, the TIX-5 sensitivity (lagtime+TIX-5/lagtime vehicle RATIO) was significantly associated with bleeding as displayed by a 1.59-fold risk of bleeding for the highest quartile (Table 1a). Moreover, while the total amount of thrombin generated (ETP) in the absence of TIX-5 did not correlate with bleeding, low ETP's measured in the presence of TIX-5 were significantly associated with bleeding (^{˜}1.63-fold increased risk for lowest 3 quartiles, Table 1.b). Thus increased sensitivity to TIX-5 of both lagtime as well as total thrombin generation is associated significantly with major bleeding during VKA anticoagulant therapy. This shows definitely clinical relevance of TIX-5 testing.

### DETAILED DESCRIPTION OF THE INVENTION

As used herein, the term “TIX-5” refers to a salivary protein antigen P23 which is capable of inhibiting factor Xa towards factor V, and having an amino acid having a sequence identity of at least 70%, more preferably 75%, 80%, 85%, 90%, 95%, 99% with the amino acid sequence having Genbank accession AEE89467.1. TIX-5 has been characterized in Circulation. 2013 Jul. 16; 128(3): 10.1161/CI RCU LATIONAHA.113.003191.

As used herein the term “bleeding” refers preferably to a major bleeding. Major bleeding events in this context are major if they are fatal, lead to a blood transfusion or hospital admission, are an intracranial bleeding, a joint bleed, or a bleeding event in a critical organ.

“Thromboembolic event” in the context of this application should be understood as the alteration of the hemostasis that leads to the development of a blood clot (thrombus) inside a vascular vessel (artery or vein). The thrombus can even obstruct the vascular vessel completely and/or become detached and obstruct another vascular vessel.

“Thromboembolic event” includes among others the following conditions: arterial thrombosis, fatal- and non-fatal myocardial infarction, stroke, transient ischemic attacks, cerebral venous thrombosis, peripheral arteriopathy, deep vein thrombosis and pulmonary embolism.

“Thromboembolic event” in the context of this application is used interchangeably with “thromboembolism”. “Thromboembolic event” in the context of this application is used interchangeably with “thrombosis”. “Thromboembolic event” in the context of this application is used interchangeably with “thromboembolic complication”.

As used herein, the term “coagulation assay” refers to any method which tests the generation of thrombin by factor Xa or the subsequent fibrin generation by thrombin as a result of factor Xa mediated thrombin generation. In a preferred embodiment, the generation of thrombin is the result of factor the Xa/factor Va complex.

The inventors have surprisingly found that TIX-5 sensitizes the plasma of patients on vitamin K antagonist (VKA) therapy, a therapy that lowers the concentration of coagulation factors, and results therefore in a hypocoaguable state which causes major bleeding in ^{˜}2% of the patients on VKA therapy.

The lowering of coagulation factors results in decreased thrombin generation, the key enzyme of coagulation. In a test that measures the time to thrombin generation (lagtime) and the total amount of thrombin generation (ETP) in a Calibrated-Automated-Thrombogram (CAT), they observed that thrombin generation was only slower (longer lagtime), and less (lower ETP) in patients with major bleeding compared to VKA treated controls without bleeding when the thrombin generation was measured in the presence of TIX-5. The thrombin generation (CAT) assay without addition of TIX-5 was not different in VKA treated patients with major bleeding or VKA treated patients without bleeding. Thus the addition of TIX-5 sensitizes the thrombin generation CAT assay to reveal a coagulation deficit that leads to major bleeding. These findings are supported by the following:
1) FIG. 9 shows that the confidence intervals (CI) of the lagtime of plasma of controls and patients with major bleeding overlap when the test is performed in the absence of TIX-5, indicating that a the test is unable to distinguish controls and patients. In contrast, the confidence intervals of controls and patients with major bleeding of the lagtime of the test performed with TIX-5 are significantly different. Thus, patients with major bleeding can be discriminated by slower thrombin generation compared to patients without bleeding, but only when thrombin generation is performed in the presence of TIX-5. This clearly shows that addition of TIX-5 sensitizes the lagtime of thrombin generation to identify a hypocoagable state that leads to major bleeding.
2) As shown in Table 1a, the hazard ratio (HR) of the lagtime in the presence of TIX-5 is statistically more significant compared to the hazard ratio (HR) of the lagtime of a test without TIX-5. It demonstrates that there is a significant association of the risk (HR 1.59 (CI 1.03-2.47)) for major bleeding in patients within the highest quartile of the TIX-5/vehicle Lagtime ratio.
3) As shown in Table 1b, the ETP (total amount of thrombin generated) determined in a test without TIX-5 does not reveal an increased risk for bleeding, while the 3 lowest quartiles of the ETP determined in the presence of TIX-5 are associated with an increased risk of major bleeding (HR's 1.66 (CI 1.06-262), 1.63 (CI 1.03-2.59) and 1.60 (CI 1.01-2.54) respectively). Thus, patients with major bleeding can be discriminated by less thrombin generation compared to patients without bleeding, but only when thrombin generation is performed in the presence of TIX-5.
4) As shown in Table 3, the predictive values for major bleeding of CAT parameters EPT and lagtime, determined in the presence of TIX-5 as estimated by Receiver Operating Characteristic curves are increased compared to predictive values of the ETP and Lagtime determined without TIX-5. In conclusion, the addition of TIX-5 sensitizes the lagtime and ETP of thrombin generation to identify a hypocoagable state that leads to major bleeding to such extend that the outcome of the thrombin generation test in the presence of TIX-5 becomes highly sensitive and therefore suitable in prediction models.

The invention therefore provides a method of determining the risk of a bleeding or a thromboembolic event in a subject comprising:
a. contacting a first sample from the subject with an activation mixture comprising (I) TIX-5, (II) factor Xa or an activation agent for activating directly or indirectly the conversion of factor X to factor Xa, and (III) a phospholipid mixture,
b. determining the value of a coagulation function parameter of said first sample,
c. comparing the value of said coagulation function parameter with a control value,
d. determining the bleeding or thromboembolic event risk based on a comparison between the value of said coagulation function parameter of said first sample and said control value.

Preferably, said sample is plasma. Preferably, venous blood is used to obtain citrated plasma via standard procedures as commonly used for Prothrombin Time (PT) and Activated Partial Thromboplastin Time (APTT) determination. In preferred embodiments, said sample comprises plasma or whole blood. In other preferred embodiments, said sample is an anticoagulant treated plasma sample. In some embodiments, the sample is selected from the group consisting of whole blood, citrated or equivalently stabilized blood, plasma, or other fluid sample containing or suspected of containing a coagulation factor. In some embodiments, the sample is whole blood. In other embodiments, the blood is venous blood. In some embodiments, the blood is fingerstick blood. In some embodiments, the sample is plasma. In some embodiments, the sample is frozen and thawed prior to contacting the sample with the activation mixture. In other embodiments, the sample has not been frozen and thawed prior to contacting the sample with the activation mixture. In some embodiments, the sample is decalcified. In some embodiments, the decalcified sample is recalcified prior to contacting the sample with the activation mixture. In other embodiments, the decalcified sample is recalcified after contacting the sample with the activation mixture.

Said first sample is contacted with an activation mixture comprising (I) TIX-5, (II) factor Xa or an activation agent for activating directly or indirectly the conversion of factor X to factor Xa, and (III) a phospholipid mixture. Said activation mixture preferably contains a suitable buffer, preferably a Hepes saline buffer of a pH around 7.35.

Said activation agent for activating directly or indirectly the conversion of factor X to factor Xa may be any compound that leads to factor Xa generation. In some preferred embodiments it is factor Xa itself. Preferably, said activation agent for activating directly or indirectly the conversion of factor X to factor Xa is selected from: Tissue factor (TF) soluble or incorporated in phospholipid vesicles, factor Vila or factor VII activators, Tissue factor/factor VII(a) complex, factor Xa or factor X activators, factor IXa or factor IX activators, factor XIIa or factor XII activators being negatively charged surfaces such as silica, kaolin, DNA, RNA, polyphosphates etc. which result in an APTT type activation of coagulation that starts with activation of the intrinsic or so-called contact system, i.e. by reciprocal activation of factor XII and prekallikrein on a negatively charged surface leading to factor XI activation by factor XIIa, Kallikrein, factor XIa or factor XI activator, cells, tissue or lysates/extracts of cells or tissues that initiate the extrinsic or intrinsic route of coagulation, platelets or activators of platelets and platelet releasate or lysate, microvesicles containing coagulation activator of any kind.

In some embodiments, the phospholipid mixture comprises 2 phospholipids. In some embodiments, the phospholipid mixture comprises 3 phospholipids. In other embodiments, the phospholipids in the phospholipid mixture are selected from the group consisting of phosphatidylcholine, phosphatidylserine, phosphatidylethanolamine, and combinations thereof.

In some embodiments, the phospholipids are natural phospholipids, synthetic phospholipids, or combinations thereof. In some embodiments, the phospholipid mixture comprises phosphatidylcholine and phosphatidylserine. In preferred embodiments, the phospholipid mixture comprises phosphatidylcholine, phosphatidylserine, and phosphatidylethanolamine. In preferred embodiments, the phospholipid mixture comprises 60% of phosphatidylcholine, 20% of phosphatidylserine, and 20% of phosphatidylethanolamine. In some embodiments, the phospholipid mixture is in lipid vesicle form. In some embodiments, the lipid vesicles are small unilamellar vesicles. In some embodiments, the activation mixture further comprises divalent cations. In other embodiments, the divalent cations are calcium ions.

The method according to the invention works best if the thrombin generation starts relatively slowly, so that the thrombin and factor Xa that must activate that factor V are subject to inhibition by fibrinogen/antithrombin and TFPI, respectively. Without wishing to be bound by theory, the inventors believe that the reason is that fibrinogen and antithrombin keep the traces of thrombin in check during the lagtime before explosive thrombin is generated once factor V is activated.

In a preferred embodiment, said activating agent is used in the coagulation assay at a concentration such that the coagulation time or lagtime is longer than 20 seconds, wherein the concentration needed to obtain a lagtime longer than 20 seconds can be easily determined by a skilled person by performing a coagulation assay with serial dilutions of the said activating agent as done for example for Tissue Factor (van't Veer et al J Biol Chem 1997; 272:4367-4377, and Schuijt et al Circulation 2013; 128:254-266). The test can then be performed with the appropriate dilution of the activating agent that generates a lagtime longer than 20 seconds. A skilled person can select the appropriate dilution for any activating agent such that the lagtime is longer than 20 seconds. Preferably, said activating agent is used in the coagulation assay at a concentration such that the coagulation time or lagtime is longer than 30, 40, 50, 60, or 100 seconds.

Said coagulation function parameter can be determined experimentally using techniques that are known in the art. In a preferred embodiment, said coagulation function parameter is selected from: coagulation time, the amount of thrombin, the lagtime, the time to peak of thrombin, the maximal velocity of thrombin generation and the thrombin peak height. These parameters are well known in the art and methods for determining them are described for instance in Hemker et al (Thromb Haemost 2006; 96:553-561).

In another preferred embodiment, said coagulation function parameter is the amount of thrombin, wherein an increase in said amount indicates a higher risk of a thromboembolic event. In a preferred embodiment, said coagulation function parameter is the lagtime, wherein an increase in lagtime indicates a higher risk of bleeding.

In a preferred embodiment, said coagulation function parameter is the coagulation time (Ct), wherein an increase of Ct compared to a control value indicates a higher risk of a bleeding.

In a preferred embodiment, said coagulation function parameter is determined by measuring the amount of thrombin. Preferably, the amount of thrombin is measured according to a standard protocol, preferably using a Fluoroskan Ascent (Thermolabsystems, Helsinki, Finnland) 96-wells fluorometer. Preferably, relipidated tissue factor is used. Preferably thrombin generation is started by addition of FluCa fluorogenic substrate/calcium solution (Trombinoscope by, STAGO) which starts the thrombin generation reaction by recalcification of the plasma. Preferably, the amount of thrombin is determined by cleavage of the fluorogenic substrate which can be detected by a fluorometer. Preferably, the amount of thrombin generated is determined by measuring the fluorescence. Preferably, thrombin generation curves are determined based on a calibrated thrombin standard. Preferably, using software, the lagtime, the maximal velocity of thrombin generation, peak height and time to peak (ttPeak), and the total amount of thrombin are generated in the sample with vehicle and with TIX-5.

In a preferred embodiment, said control value is a Ct obtained from a corresponding standard. Suitable standards include normal pool plasma or a reference plasma of VKA treated patients.

It is further preferred that said coagulation time is determined by measuring the time between the contacting of the activation mixture with said first sample and the onset of coagulation, thereby calculating the coagulation time (Ct). In a further preferred embodiment, the method of the invention further comprises:

a. contacting a second sample from the subject with an activation mixture comprising (I) factor Xa or an activation agent for activating directly or indirectly the conversion of factor X to factor Xa, and (II) a phospholipid mixture and (III) without TIX-5,

b. measuring the time between the contacting of the activation mixture with said second sample and the onset of coagulation, thereby calculating the coagulation time of said second sample (Ct2), wherein an increase in Ct compared to Ct2 indicates a higher risk of a bleeding or thromboembolic event.

Preferably, said comparison comprises determining the ratio between Ct and Ct2, wherein an increase in the ratio between Ct and Ct2 indicates a higher risk of a bleeding or a thromboembolic event.

In another preferred embodiment, said coagulation function parameter is the amount of thrombin, wherein said risk of bleeding is indicated when the amount of thrombin in said first sample is lower than a thrombin control value. Said control value may be a predetermined reference value or the amount of thrombin in standard normal pool plasma or a reference plasma of VKA treated patients. In other preferred embodiments, said control value is based on one or multiple control samples from healthy individuals. Preferably said control value is based on normal pool plasma.

In a preferred embodiment of the method according to the invention said an activation agent is Tissue Factor (TF). Preferably, said activation agent has a concentration of more than 5, 10 or 20 pM.

It is preferred that said subject is in need of an anti-coagulant therapy. In a preferred embodiment, said subject is a patient treated using an anticoagulant therapy, preferably a vitamin K antagonist anticoagulant therapy (VKA).

The invention further provides a kit which is suitable for carrying out the method of the invention, comprising:

a. TIX-5,

b. Tissue Factor in a concentration of 10 pM or higher, and

c. a phospholipid mixture.

The invention further provides the use of TIX-5 in a coagulation assay on an anticoagulant treated plasma sample. Said assay is preferably a CAT assay.

The invention is more closely illustrated by means of the following examples.

### Example

Material and Methods

Purified TIX-5

Highly purified recombinant TIX-5 for the studies herein was obtained by purification of TIX-5 produced in the Drosophila Expression System (Invitrogen) using the pMT/Bip/V5-HisA plasmid as described before (Schuijt et al, Circulation 2013; 128:254-266). Briefly, supernatant medium of S2 Drosophila cells producing TIX-5 was loaded on a Ni-NTA Superflow column and bound TIX-5 was eluted with imidazole. TIX-5 containing fractions were pooled and cleared by passing over DEAE-Sepharose at 100 mM NaCl. The TIX-5 in the flow through was concentrated on a SP-Sepharose column that was equilibrated with 25 mM MES, pH 6.5, 100 mM NaCl, washed and eluted with MES buffer and 1 M NaCl. The eluted TIX-5 peak was concentrated for final cleanup and buffer exchange on a size exclusion S200 26/600 column (GE Healthcare) equilibrated in PBS. TIX-5 containing fractions eluted from the S200 column were concentrated and frozen in aliquots at −80° C. The thus obtained highly purified TIX-5 preparation was >99% pure as judged by SDS-PAGE and Coomassie blue staining. Protein concentration was estimated by A280 measurement using an extinction coefficient of 0.4 and a molecular weight of 30 kD. Prepared this way different TIX-5 lots inhibited TF (5 pM) initiated thrombin generation identical at 4 μM in normal pool plasma, and did not affect thrombin (3 nM) initiated FXI dependent thrombin generation in normal pool plasma at 4 μM. The latter is in line with the specific mechanism of action of TIX-5 on the activation of FV by FXa as previously described and lack of effect on FV activation by thrombin (Schuijt et al, Circulation 2013; 128:254-266).

Citrated Plasma

Thrombin generation was performed on citrated platelet poor plasma obtained by centrifugation of venous blood collected by venapunture in citrate tubes by standard procedures as commonly used for Prothrombin Time (PT) and Activated Partial Thromboplastin Time (APTT) determination. Normal pool plasma was prepared by pooling the citrate plasma of 60 healthy volunteers via standard procedures used to make a reference normal sample for coagulation tests as known in the art and stored in aliqouts at −80° C. Individual plasma samples of 60 blood donors that donated blood for the creation of normal pool plasma was also stored at −80° C. to investigate differences in healthy persons. Citrated plasma of patients on vitamin K antagonist (VKA) therapy was stored as part of the BLEEDS study described in van Rein et al, PLoS ONE 2016; 11:e0164485. In the BLEEDS study plasma was collected of patients after 3 weeks of start with VKA anticoagulant therapy and major bleeding while under VKA therapy was monitored. Incidence rate of major bleeding was 1.85/100 person years. More than 16.000 patients on VKA were included in the BLEEDS study which was powered to find biomarkers for the risk of major bleeding during anticoagulant therapy. Plasma of 244 cases with major bleeding of the BLEEDS study were compared to 538 randomly selected samples of the BLEEDS study (subcohort) to estimate a potential association of measured parameters in the plasma with major bleeding.

Thrombin Generation

Thrombin generation in plasma samples was performed using the Calibrated Automated Thrombogram (CAT) method with the Thrombinoscope reagents as supplied by Stago (Leiden, The Netherlands). Tissue factor (TF) initiated CAT was performed in platelet poor plasma of healthy human volunteers or the normal pool of these with 5 pM TF. CAT in plasma of patients on VKA was initiated with 20 pM TF to overcome the anticoagulant effect in the latter. In short, plasma was thawed for 15 min at 37° C. and 80 μL plasma aliquots were transferred to an Immulon 2HB round-bottom 96-well plate (Thermo Scientific), and mixed with 20 μL PPP reagent, consisting of a phospholipid vesicle mixture (20% PS:20% PE:60% PC, final concentration 4 μM) and the appropriate amount of TF. Then either TIX-5 (4 μM final concentration) was added in 10 μL PBS to the mixture, or only 10 μL PBS was added as vehicle control in the uninhibited reaction samples. After 10 min of incubation at 37° C. in a Fluoroskan Ascent microtiter plate-reading fluorometer (Thermo Labsystems), the thrombin generation reaction was initiated with 20 μL of calcium/fluorogenic substrate reagent (FluCa Kit, Stago). Thrombin generation was assessed by detection of the fluorescent signal using 390 nm excitation and 460 nm emission filters for 60 min with 20 s intervals. The fluorescent signal was converted to thrombin levels using Thrombinoscope software according to the manufacturer's instructions (Hemker H C, Calibrated automated thrombinography (CAT). Thromb. Res. 2005; 115, 255) and with Thrombin Calibrator (Stago) as calibration standard. The following parameters were analyzed by the Thrombinoscope Software: lag time in min, endogenous thrombin potential (ETP) in nM*min, peak in nM, time to peak (ttPeak) in min and velocity in nM/min.

Statistical Analysis

Comparisons between groups were tested by using the (non-parametric) Mann-Whitney U-test. Comparisons between the vehicle control and the TIX-5 data for the same individual were performed by using the Wilcoxon matched-pairs ranked test. Analyses were made using GraphPad Prism version 5.01 (GraphPad Software, San Diego, Calif., USA). p values <0.05 were considered statistically significant. Hazard ratios (HR) and 95% confidence intervals (95% CI) were estimated after dividing the data into quartiles or tertiles (as observed in controls without complication) by means of Cox regression in R (version 3.2.0, R Foundation for Statistical Computing, Vienna, Austria). Receiver operating characteristic (ROC) area under the curve (AUC) analysis with significance of predictive value was analyzed using the pROC package (Robin X et al, BMC Bioinformatics, 2011; 12:77).

In FIG. 9 the result is shown of the TIX-5 CAT assay initiated with 20 pM TF on stored plasma's of a randomly chosen subcohort of the BLEEDS study (538 plasma's) and compared those to plasmas of the cases with major bleeding (244 patients) while under VKA anticoagulant therapy. Lagtimes of thrombin generation in the presence of TIX-5 (white bars) are significantly longer in patients that displayed major bleeding (FIG. 9) which translates in a significant 2.05-fold increased risk for bleeding (Table 1a) in the quartile with the highest lagtime+TIX-5. Furthermore, the TIX-5 sensitivity (lagtime+TIX-5/lagtime vehicle RATIO) was significantly associated with bleeding as displayed by a 1.59-fold risk of bleeding for the highest quartile (Table 1a). Moreover, while the total amount of thrombin generated (ETP) in the absence of TIX-5 did not correlate with bleeding, low ETP's measured in the presence of TIX-5 were significantly associated with bleeding (^{˜}1.63-fold increased risk for the lowest 3 quartiles, Table 1.b). Thus increased sensitivity to TIX-5 of both lagtime as well as total thrombin generation is associated significantly with major bleeding during VKA anticoagulant therapy. This shows definitely clinical relevance of TIX-5 testing. For 60 cases with thrombotic events despite VKA treatment in the BLEEDS study no association was observed of any of the separate thrombin generation parameters with or without TIX-5 (Table.2), however TIX-5 sensitivity of the lagtime of the highest tertile was associated with a 2.14-fold increased risk of a thrombotic complication.

Hazard ratio's and 95% confidence intervals for major bleeding and thrombosis of the 16.000 patients in the BLEEDS study with respect to parameters of the TIX-5 assay. (*P<0.05, **P<0.01)

TABLE 1a
BLEEDING

| | |
|---|---|
| Cases | HR (95% CI) |

Lagtime vehicle

| <25 | (<4.32) | 44 | reference | |
|---|---|---|---|---|
| 25-50 | (4.32-5.67) | 64 | 1.54 | (0.97-2.47) |
| 50-75 | (5.67-7.29) | 52 | 1.14 | (0.71-1.85) |
| >75 | (>7.29) | 84 | 2.02 | (1.29-3.17)* |

Lagtime + TIX-5

| <25 | (<8.00) | 45 | reference | |
|---|---|---|---|---|
| 25-50 | (8.00-10.99) | 50 | 1.19 | (0.74-1.94) |
| 50-75 | (11.00-15.66) | 65 | 1.48 | (0.93-2.36) |
| >75 | (>15.67) | 84 | 2.05 | (1.31-3.22)** |

TIX-5/vehicle Lagtime ratio

| <25 | (<1.56) | 56 | reference | |
|---|---|---|---|---|
| 25-50 | (1.56-1.95) | 59 | 1.05 | (0.67-1.66) |
| 50-75 | (1.95-2.50) | 53 | 1.06 | (0.67-1.68) |
| >75 | (>2.50) | 76 | 1.59 | (1.03-2.47)* |

TABLE 1b
BLEEDING

| | |
|---|---|
| Cases | HR (95% CI) |

ETP vehicle

| | | | | |
|---|---|---|---|---|
| <25 | (<384) | 74 | 1.37 | (0.89-2.11) |
| 25-50 | (384-531) | 59 | 1.11 | (0.71-1.75) |
| 50-75 | (531-730) | 54 | 0.94 | (0.59-1.48) |

| | | | |
|---|---|---|---|
| >75 | (>730) | 57 | reference |

ETP TIX-5

| | | | | |
|---|---|---|---|---|
| <25 | (<305) | 69 | 1.66 | (1.06-2.62)* |
| 25-50 | (305-438) | 64 | 1.63 | (1.03-2.59)* |
| 50-75 | (438-637) | 64 | 1.60 | (1.01-2.54)* |

| | | | |
|---|---|---|---|
| >75 | (>637) | 47 | reference |

TIX-5/vehicle ETP ratio

| | | | | |
|---|---|---|---|---|
| <25 | (<0.76) | 67 | 1.38 | (0.89-2.16) |
| 25-50 | (0.76-0.85) | 50 | 0.96 | (0.60-1.53) |
| 50-75 | (0.85-0.94) | 72 | 1.24 | (0.80-1.92) |

| | | | |
|---|---|---|---|
| >75 | (>0.94) | 55 | reference |

TABLE 2a
THROMBOSIS

| | |
|---|---|
| Cases | HR (95% CI) |

Lagtime vehicle

| <33 | (<4.67) | 20 | reference | |
|---|---|---|---|---|
| 33-67 | (4.67-6.67) | 20 | 0.94 | (0.47-1.91) |
| >67 | (>6.67) | 20 | 1.03 | (0.50-2.10) |

Lagtime TIX-5

| <33 | (<9.00) | 21 | reference | |
|---|---|---|---|---|
| 33-67 | (9.00-13.52) | 19 | 0.84 | (0.41-1.70) |
| >67 | (>13.52) | 20 | 1.11 | (0.55-2.26) |

TIX-5/vehicle Lagtime ratio

| <33 | (<1.69) | 15 | reference | |
|---|---|---|---|---|
| 33-67 | (1.69-2.32) | 24 | 1.58 | (0.76-3.27) |
| >67 | (>2.32) | 21 | 2.14 | (1.01-4.54)* |

TABLE 2b
THROMBOSIS

| | |
|---|---|
| Cases | HR (95% CI) |

ETP vehicle

| | | | | |
|---|---|---|---|---|
| <33 | (<430) | 20 | 1.46 | (0.70-3.04) |
| 33-67 | (430-658) | 23 | 1.71 | (0.83-3.51) |

| | | | |
|---|---|---|---|
| >67 | (>658) | 17 | reference |

ETP TIX-5

| | | | | |
|---|---|---|---|---|
| <33 | (<347) | 17 | 1.20 | (0.57-2.54) |
| 33-67 | (347-565) | 25 | 1.58 | (0.79-3.16) |

| | | | |
|---|---|---|---|
| >67 | (>565) | 18 | reference |

TIX-5/vehicle ETP ratio

| | | | | |
|---|---|---|---|---|
| <33 | (<0.79) | 19 | 1.11 | (0.53-2.30) |
| 33-67 | (0.79-0.92) | 22 | 1.04 | (0.52-2.10) |

| | | | |
|---|---|---|---|
| >67 | (>0.92) | 19 | reference |

To show the predictive value for bleeding of the thrombin generation assay with TIX-5 compared to the thrombin generation without TIX-5, we evaluated risk predicting models using Receiver Operating Characteristic (ROC) curves generated on the basis of our BLEEDS subcohort study as described above. The area under the curve (AUC) and predictive P-value for bleeding of the thrombin generation parameters with and without TIX-5 are shown in Table 3 below. As can be observed the lagtime of thrombin generation measured in the presence of TIX-5 is a superior parameter with regard to predictive value for major bleeding. The parameters measured in the absence of TIX-5 have much lower or no predictive value.

TABLE 3
Receiver operating characteristic area under the curve
(ROC AUC) analysis to estimate the predictive value of
CAT parameters with and without TIX-5 for major bleeding
in the BLEEDS study. Standard error (se) and P-value.

| Marker (univariate) | ROC AUC (se) for BLEEDING | P-value |
|---|---|---|
| ETP + TIX-5 | 0.554 (0.02) | 0.0283 |
| Lagtime + TIX-5 | 0.584 (0.02) | 0.0000864 |
| ETP + vehicle | 0.542 (0.02) | 0.0871 |
| Lagtime + vehicle | 0.551 (0.02) | 0.0762 |

1. A method of determining the risk of a bleeding or a thromboembolic event in a subject comprising:
a. contacting a first sample from the subject with an activation mixture comprising (I) TIX-5, (II) factor Xa or an activation agent for activating directly or indirectly the conversion of factor X to factor Xa, and (III) a phospholipid mixture,
b. detecting a coagulation function parameter of said first sample and determining a value therefor,
c. comparing the value of said coagulation function parameter with a control value,
d. determining said risk based on a comparison between the value of said coagulation function parameter of said first sample and said control value.

2. The method according to claim 1, wherein said coagulation function parameter is selected from: the amount thrombin, coagulation time, the lagtime, the time to peak of thrombin, the maximal velocity of thrombin generation and the thrombin peak height.

3. The method according to claim 2, wherein said coagulation function parameter is the coagulation time (Ct), wherein an increase of Ct compared to a control value indicates a higher risk of a bleeding or thromboembolic event.

4. The method according to claim 2, wherein said coagulation time is determined by measuring the time between the contacting of the activation mixture with said first sample and the onset of coagulation, thereby calculating the coagulation time (Ct).

5. The method according to claim 1, wherein said coagulation function parameter is the amount of thrombin, wherein a risk of bleeding or thromboembolic event is indicated when the amount of thrombin in said first sample is lower than a thrombin control value.

6. The method according to claim 1, further comprising:
a. contacting a second sample from the subject with an activation mixture comprising (I) factor Xa or an activation agent for activating directly or indirectly the conversion of factor X to factor Xa, and (II) a phospholipid mixture and (III) without TIX-5,
b. measuring the time between the contacting of the activation mixture with said second sample and the onset of coagulation, thereby calculating the coagulation time of said second sample (Ct2), wherein an increase in Ct compared to Ct2 indicates a higher risk of a bleeding or thromboembolic event.

7. The method according to claim 1, wherein said comparison comprises determining the ratio between Ct and Ct2, wherein an increase in the ratio between Ct and Ct2 indicates a higher risk of bleeding or thromboembolic event.

8. The method according to claim 1, wherein said activation agent is Tissue Factor (TF).

9. The method according to claim 1, wherein said activation agent has a concentration of more than 5, 10 or 20 pM.

10. The method according to claim 1, wherein said subject is in need of an anti-coagulant therapy.

11. The method according to claim 1, wherein said subject is a patient treated using an anticoagulant therapy.

12. The method according to claim 10, wherein said anticoagulant therapy is a vitamin K antagonist anticoagulant therapy (VKA).

13. The method according to claim 1, wherein said sample comprises plasma or whole blood.

14. A kit for carrying out the method according to claim 1, comprising:
a. TIX-5,
b. TF in a concentration of 10 pM or higher, and
c. a phospholipid mixture.

15. A product comprising TIX-5 in a coagulation assay on a plasma sample from a subject treated with a vitamin K antagonist.
